(19) United States
(12) Patent Application Publication
(10) Pub. No.: US 2005/0261520 A1
(43) Pub. Date: Nov. 24, 2005

(54) Process for producing catalyst for production of unsaturated carboxylic acid

(75) Inventors: Isao Teshigahara, Mie (JP); Kazuharu Tazawa, Mie (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: MITSUBISHI CHEMICAL CORPORATION, Tokyo (JP)

(21) Appl. No.: 11/032,050

(22) Filed: Jan. 11, 2005

Related U.S. Application Data
(63) Continuation of application No. PCT/JP04/13463, filed on Sep. 15, 2004.

(30) Foreign Application Priority Data
May 21, 2004 (JP) 2004-151974

(51) Int. Cl. C07C 51/16; C07C 51/235

(52) U.S. Cl. 562/547

(57) ABSTRACT

A new process for producing a catalyst which has a high mechanical strength and which is capable of producing an unsaturated carboxylic acid constantly over a long period of time and in good yield, is presented. A process for producing a catalyst for the production of an unsaturated carboxylic acid, which is a process for producing a catalyst containing at least molybdenum and vanadium, to be used at the time of gas phase catalytic oxidation of an unsaturated aldehyde with a molecular oxygen-containing gas to produce the unsaturated carboxylic acid, characterized in that a mixed solution or aqueous slurry containing the above catalyst components, is dried, the moisture content of the obtained dried product is adjusted to be from 0.5 to 4 wt %, and the powder thus adjusted is subjected to tabletting to obtain a molded product.

### TECHNICAL FIELD

The present invention relates to a process for producing a catalyst which is a catalyst for gas phase catalytic oxidation of an unsaturated aldehyde with a molecular oxygen-containing gas to produce the corresponding unsaturated carboxylic acid, and which has a high mechanical strength and is capable of producing the corresponding unsaturated carboxylic acid constantly over a long period of time and in good yield.

### BACKGROUND ART

Heretofore, various proposals have been made with respect to catalysts for gas phase catalytic oxidation of an unsaturated aldehyde such as acrolein or methacrolein with a molecular oxygen-containing gas to produce the respectively corresponding unsaturated carboxylic acid such as acrylic acid or methacrylic acid.

These catalysts are, of course, required to be able to produce the desired unsaturated carboxylic acid in good yield, but also required to have sufficiently high mechanical strength and durability durable for industrial use for a long period of time.

Heretofore, in order to improve the properties of the catalyst to be used for such a reaction, it has been proposed to control pores of the catalyst by using various organic compounds at the time of the preparation of the catalyst. As such examples, Patent Documents 1 and 2, etc. are known.

Patent Document 1 is to improve the catalyst performance by controlling the pore structure of a catalyst in such a manner that a polymer organic compound having a specific particle size is added at the time of molding a catalyst, and the added polymer organic compound is removed by heat treatment. However, in this method, at the time of removing the polymer organic compound by heat treatment, sintering of the catalyst by the combustion of the organic compound or reduction of the catalyst by the polymer organic compound, is likely to take place, whereby not only the activating treatment is complicated, but also there will be a problem such that reproducibility of the catalyst performance is poor.

Whereas, Patent Document 2 is to improve the catalyst performance by improving the moldability of the catalyst by molding a catalyst component having an activated carbon powder having an average particle size of from 1 to 500 μm added. However, also in this case, there still is a room for improvement in the yield of the desired unsaturated carboxylic acid, and it is desired to further improve or increase the catalyst performance.

Patent Document 1: JP-A-5-192588

Patent Document 2: JP-A-6-374

### DISCLOSURE OF THE INVENTION

In view of the above-described prior art, it is an object of the present invention to provide a process for producing a catalyst which is capable of gas phase catalytic oxidation of an unsaturated aldehyde with a molecular oxygen-containing gas to produce the corresponding unsaturated carboxylic acid in good yield and with good reproducibility and which also has high mechanical strength and high durability.

### MEANS TO SOLVE THE PROBLEM

The present inventors have conducted an extensive research to solve the above problem and have found that in the production of a composite oxide catalyst containing at least molybdenum and vanadium, to produce an unsaturated carboxylic acid from an unsaturated aldehyde, the amount of moisture contained in the dried product obtained by drying a mixed solution or slurry containing catalyst components, followed by molding, is largely influential over the characteristics such as the activity, mechanical strength, etc. of the catalyst, and it is possible to solve the above problem by controlling it within a proper range.

Namely, by the study made by the present inventors, it has been found that a molded catalyst whereby the desired unsaturated carboxylic acid can be produced in good yield, and the mechanical strength is high and constant, can be obtained by molding while controlling the amount of moisture contained in a dried product obtained from a mixed solution or slurry containing the catalyst components, to a level of from 0.5 to 4 wt %, as will be seen in Examples and Comparative Examples given hereinafter. If the moisture content in the above dried product is smaller than 0.5 wt %, the molding pressure required to obtain good characteristics tends to be high, and it tends to be difficult to obtain a molded product of the catalyst having constant characteristics. On the other hand, in the case of a dried product having a moisture content of larger than 4 wt %, the characteristics of the catalyst tend to change and the mechanical strength tends to decrease in the calcining step following the molding, whereby it tends to be difficult to obtain a catalyst having a high performance.

Thus, the present invention is characterized by the following constructions.

(1) A process for producing a catalyst for the production of an unsaturated carboxylic acid, which is a process for producing a composite oxide catalyst containing at least molybdenum and vanadium, to be used at the time of gas phase catalytic oxidation of an unsaturated aldehyde with a molecular oxygen-containing gas to produce an unsaturated carboxylic acid, characterized in that a mixed solution or aqueous slurry containing the above catalyst components, is dried, the moisture content of the obtained dried product is adjusted to from 0.5 to 4 wt %, and the obtained powder is tabletted to obtain a molded product.

(2) The process for producing a catalyst for the production of an unsaturated carboxylic acid according to the above (1), wherein the molded product is of a ring shape opened in the longitudinal direction and having an outer diameter of from 3 to 10 mm, an inner diameter being from 0.1 to 0.7 time the outer diameter and a length being from 0.5 to 2 times the outer diameter.

(3) The process for producing a catalyst for the production of an unsaturated carboxylic acid according to the above (1) or (2), wherein the composite oxide catalyst has the following formula (1):
MoaVbSbcNbdNieXfYgOh (1)
(wherein X is at least one element selected from the group consisting of Si and Al, Y is at least one element selected from the group consisting of Cu and W, and a to h represent atomic ratios of the respective elements, and when a=12, b=0.1 to 10, c=1 to 100, d=0 to 10, e=1 to 100, f=0 to 200, and g=0.1 to 10, and h is a numerical value which satisfies the oxidized states of other elements.)
(4) A method for producing an unsaturated carboxylic acid, which comprises gas phase catalytic oxidation of an unsaturated aldehyde with a molecular oxygen-containing gas by using a catalyst produced by the process as defined in any one of the above (1) to (3), to produce the corresponding unsaturated carboxylic acid.
(5) The method according to the above (4), wherein the unsaturated aldehyde is acrolein, and the unsaturated carboxylic acid is acrylic acid.

### EFFECTS OF THE INVENTION

According to the process of the present invention, it is possible to provide a catalyst which has a high mechanical strength and which is capable of gas phase catalytic oxidation of an unsaturated aldehyde with a molecular oxygen-containing gas to produce an unsaturated carboxylic acid, constantly over a long period of time and in good yield, by a means based on a new concept such that at the time of producing a composite oxide catalyst containing at least molybdenum and vanadium, tabletting is carried out while controlling the amount of moisture contained in the dried product obtained by drying a mixed solution or slurry containing the catalyst components, followed by molding, within a specific range.

### BEST MODE FOR CARRYING OUT THE INVENTION

The catalyst to be produced by the present invention, is a catalyst for gas phase catalytic oxidation of an unsaturated aldehyde such as acrolein or methacrolein with a molecular oxygen-containing gas to produce the respectively corresponding unsaturated carboxylic acid such as acrylic acid or methacrylic acid and is a composite oxide catalyst containing at least molybdenum and vanadium. The present invention is applicable to any catalyst so long as it is a composite oxide catalyst containing such two components. However, it is particularly preferably applicable to a catalyst represented by the following formula (1):
MoaVbSbcNbdNieXfYgOh (1)

In the formula (1), Mo represents molybdenum, V vanadium, Sb antimony, Nb niobium, Ni nickel and O oxygen, and X, Y, a, b, c, d, e, f, g and h are as defined above. Especially, it is particularly preferred that when a=12, b=1 to 5, c=10 to 50, d=0 to 5, e=10 to 50, f=1 to 100 and g=1 to 5.

In the process of the present invention, raw material compounds containing the respective element components of the catalyst are suitably dissolved or dispersed in an aqueous medium, in amounts required depending upon the composition of the catalyst to be produced, to obtain a mixed solution or aqueous slurry containing the catalyst components. The raw materials for the respective catalyst components may, for example, be nitrates, ammonium salts, hydroxides, oxides, sulfates, carbonates, halides or acetates of the respective elements. For example, for molybdenum, ammonium paramolybdate, molybdenum trioxide or molybdenum chloride may be used. To the aqueous medium, a non-aqueous solvent such as an alcohol may be added in order to adjust the viscosity, as the case requires.

The mixed solution or aqueous slurry containing the catalyst components, is preferably thoroughly stirred and mixed in order to prevent localization of each component. Then, the mixed solution or aqueous slurry containing the catalyst components is dried. The drying may be carried out by various methods. For example, a spray dryer, slurry dryer or drum dryer may be used to obtain a powdery dried product. However, drying by means of a spray dryer is particularly preferred.

In the present invention, it is important to control the moisture content of the dried product preferably in a powder form, which is to be subjected to molding and which contains the catalyst components. As mentioned above, the moisture content of the dried product is adjusted to be from 0.5 to 4 wt %. The moisture content of the dried product is defined by the formula (2).

In the formula (2), W1 is the weight when the dried product is subjected to evaporation to dryness at 150° C. for 10 hours, and W2 is the weight of the dried product.
Moisture content=(W2-W1)/W1×100 (2)

In the present invention, as the method to adjust the moisture content of the dried product to be subjected to molding, to be within the above range, the conditions for drying the mixed solution or aqueous slurry containing the catalyst components may be controlled, or proper humidification may be applied by a method of e.g. spraying moisture to the dried product once produced. In any case, the moisture content of the dried product is required to be adjusted to be within the above range. Especially, in the present invention, the moisture content of the dried product is preferably from 0.5 to 4 wt %, more preferably from 0.7 to 3.5 wt %, particularly preferably from 1 to 3 wt %.

As the molding method of the dried product, in the present invention, a tabletting method is employed from the viewpoint of the efficiency in molding and the nature of the molded product. The shape of the product may be any shape such as a spherical, cylindrical or ring-shape. Further, with respect to the size, various sizes may suitably be selected. However, it is particularly preferred that the molded product is of a ring-shape opened in a longitudinal direction and having an outer diameter of from 3 to 10 mm, an inner diameter being from 0.1 to 0.7 time the outer diameter and a length being from 0.5 to 2 times the outer diameter.

At the time of the above molding, in order to improve the mechanical strength or degradation of the molded product, commonly known inorganic fibers such as glass fibers, various whiskers, etc. may be used. Further, in order to control the physical properties of the catalyst to have good reproducibility, an additive which is commonly known as a binder, such as ammonium nitrate, cellulose, starch, polyvinyl alcohol or stearic acid, may also be used.

In the present invention, the molded product of the dried product containing the catalyst components thus obtained, is then calcined. The calcining is preferably carried out in the presence of an oxygen-containing gas preferably at from 300 to 500° C., particularly preferably at from 350 to 450° C., preferably for from 1 to 15 hours, particularly preferably for from 3 to 12 hours. For such calcining, an atmospheric calcining furnace may be employed. As such an atmospheric calcining furnace, there may, for example, be employed a method wherein the catalyst is packed in a fixed bed reactor and heating is carried out from the exterior while circulating an atmospheric gas, a method wherein the above fixed bed reactor is of a heat exchange type, a method wherein an atmospheric gas is circulated in the interior of a muffle furnace, a method wherein an atmospheric gas is circulated into the interior of a tunnel furnace, or a method wherein an atmospheric gas is circulated in the interior of a kiln furnace. Taking the efficiency for control of the atmospheric gas flow rate in the calcining into consideration, it is preferred to employ a method wherein the catalyst is packed in a fixed bed reactor and heating is carried out from the exterior while circulating an atmospheric gas, more preferably a method wherein the catalyst is packed into a heat exchange type fixed bed reactor, and heating is carried out from the exterior while circulating an atmospheric gas. As the atmospheric gas, not only air, but a mixed gas of inert gasses, such as air and nitrogen, may be employed. From the economical advantage, it is preferred to employ air.

A method for gas phase catalytic oxidation of an unsaturated aldehyde with a molecular oxygen-containing gas by means of the catalyst produced by the present invention to produce the corresponding unsaturated carboxylic acid, may be carried out by a conventional method. For example, as the reactor, a fixed bed tubular reactor may be employed. In such a case, the reaction may be a single flow process through the reactor, or may be a recycle process, and it may be carried out under such conditions as commonly employed in a reaction of this type.

For example, a mixed gas comprising from 1 to 15 vol % of acrolein, from 0.5 to 25 vol % of molecular oxygen, from 0 to 40 vol % of steam, from 20 to 80 vol % of an inert gas such as nitrogen or carbon dioxide gas, is introduced to a catalyst layer having the catalyst packed in each reaction zone of each reaction tube having an internal diameter of preferably from 15 to 50 mm at from 200 to 400° C. under a pressure of from 0.1 to 1 MPa at a space velocity (SV) of from 300 to 5000 hr⁻¹. In the present invention, in order to increase the productivity, the operation may be made under a higher load reaction condition, such as a higher raw material concentration or a higher space velocity. Thus, by the catalyst produced by the present invention, it is possible to produce acrylic acid with high selectivity and in good yield.

### EXAMPLES

Now, the present invention will be described in further detail with reference to Examples of the present invention. However, it should be understood that the present invention is by no means restricted to such Examples. In the following, the conversion, the selectivity and the yield are calculated by the following formulae. Further, the drop impact strength of the catalyst is one obtained as described below.

Conversion of acrolein (mol %): (mols of reacted acrolein/mols of supplied acrolein)×100

Selectivity (mol %): ((mols of formed acrylic acid)/mols of reacted acrolein)×100

Yield (mol %): ((mols of formed acrylic acid)/mols of supplied acrolein)×100

Drop impact strength: In a stainless steel pipe having an inner diameter of 25 mm and a length of 1 m, which was vertically set, 20 g of a catalyst was dropped from the top and received on a stainless steel plate having a thickness of 2 mm, whereupon the weight of the catalyst remaining on the plate was measured, followed by sieving with a sieve of 10 mesh. The drop impact strength was obtained by the following formula.

Drop impact strength (%)=(weight of the catalyst remaining on the sieve/weight of the catalyst dropped)×100

### Example 1

60 g of basic nickel carbonate (Ni content: 43%) was dispersed in 300 ml of pure water, and 50 g of silica (“Carplex #67”) and 150 g of antimony trioxide were added thereto, followed by sufficient stirring. The obtained slurry was heated, concentrated and dried. Then, the obtained solid was calcined at 800° C. for 3 hours in a muffle furnace, and the calcined product was pulverized to obtain a powder of at most 60 mesh.

On the other hand, 540 ml of pure water was heated to about 80° C., and 8.1 g of ammonium paratungstate, 63.9 g of ammonium paramolybdate, 8.4 g of ammonium methavanadate and 3.1 g of cuprous chloride were sequentially added and dissolved with stirring. To this solution, the above powder was added, followed by sufficient stirring and mixing to obtain a slurry.

This slurry was heated to from 80° C. to 100° C. and then dried in a spray dryer under such conditions that the inlet temperature was 290° C. and the outlet temperature was 125° C. The moisture content of the obtained dried product was 2.0 wt %.

To this dried product, 1.5 wt % of graphite was added, and the mixture was molded by a tabletting machine into ring-shaped tablets having an outer diameter of 6 mm, an inner diameter of 3 mm and a height of 4 mm. The molded product was calcined at 400° C. for 5 hours in a muffle furnace to obtain a catalyst. The composition of the obtained catalyst was as follows by atomic ratio (excluding O). The drop impact strength of the catalyst was measured and found to be 98%.

Sb:Ni:Si:Mo:V:W:Cu=34:15:27:12:2.4:1:1

30 ml of the above catalyst was packed into a stainless steel reaction tube equipped with a niter jacket and having an inner diameter of 20 mm and a length of 500 mm, and a mixed gas contained by catalytic oxidation of propylene in the presence of a molybdenum/bismuth oxide type multi-element catalyst, was introduced and passed through this reaction tube at a space velocity of 1,300 hr⁻¹ on 0° C. standard, to carry out the reaction at a temperature of 260° C. Here, the average composition of the mixed gas introduced, was as follows.
Acrolein: 5.95%
Propylene+propane: 0.16%
Acrylic acid+Acetic acid: 0.77%
Oxygen: 7.55%
Nitrogen: 62.41%
Steam: 22.55%
Others: 0.61%

As a result of the reaction, the conversion of acrolein was 99.2° C., the selectivity was 98.1%, and the yield was 97.3%.

### Comparative Example 1

A catalyst was prepared in the same manner as in Example 1 except that the outlet temperature of the spray dryer was changed to 160° C. The moisture content of the dried product was 0.4 wt %. The drop impact strength of this catalyst was measured and found to be 90.1%.

An oxidation reaction was carried out in the same manner as in Example 1, whereby the conversion of acrolein was 98.1%, the selectivity was 97.9%, and the yield was 96.0%.

### Comparative Example 2

A catalyst was prepared in the same manner as in Example 1 except that the outlet temperature of the spray dryer was changed to 95° C. The moisture content of the dried product obtained, was 5.3 wt %. The drop impact strength of this catalyst was measured and found to be 91.5%.

An oxidation reaction was carried out in the same manner as in Example 1, whereby the conversion of acrolein was 97.8%, the selectivity was 97.7%, and the yield was 95.6%.

### INDUSTRIAL APPLICABILITY

The catalyst produced by the process of the present invention, is useful for gas phase catalytic oxidation of an unsaturated aldehyde with a molecular oxygen-containing gas to produce the corresponding unsaturated carboxylic acid. The produced unsaturated carboxylic acid is useful in a wide range of applications as e.g. a starting material for various chemical products, a monomer for common resins, a monomer for functional resins such as water absorptive resins, a flocculating agent or a thickener.

The entire disclosure of Japanese Patent Application No. 2004-151974 filed on May 21, 2004 including specification, claims and summary is incorporated herein by reference in its entirety.

1. A process for producing a catalyst for the production of an unsaturated carboxylic acid, which is a process for producing a composite oxide catalyst containing at least molybdenum and vanadium, to be used at the time of gas phase catalytic oxidation of an unsaturated aldehyde with a molecular oxygen-containing gas to produce an unsaturated carboxylic acid, characterized in that a mixed solution or aqueous slurry containing the above catalyst components, is dried, the moisture content of the obtained dried product is adjusted to from 0.5 to 4 wt %, and the obtained powder is tabletted to obtain a molded product.

2. The process for producing a catalyst for the production of an unsaturated carboxylic acid according to claim 1, wherein the molded product is of a ring shape opened in the longitudinal direction and having an outer diameter of from 3 to 10 mm, an inner diameter being from 0.1 to 0.7 time the outer diameter and a length being from 0.5 to 2 times the outer diameter.

3. The process for producing a catalyst for the production of an unsaturated carboxylic acid according to claim 1, wherein the composite oxide catalyst has the following formula (1):
MoaVbSbcNbdNieXfYgOh (1)
(wherein X is at least one element selected from the group consisting of Si and Al, Y is at least one element selected from the group consisting of Cu and W, and a to h represent atomic ratios of the respective elements, and when a=12, b=0.1 to 10, c=1 to 100, d=0 to 10, e=1 to 100, f=0 to 200, and g=0.1 to 10, and h is a numerical value which satisfies the oxidized states of other elements.)

4. A method for producing an unsaturated carboxylic acid, which comprises gas phase catalytic oxidation of an unsaturated aldehyde with a molecular oxygen-containing gas by using a catalyst produced by the process as defined in claim 1, to produce the corresponding unsaturated carboxylic acid.

5. The method according to claim 4, wherein the unsaturated aldehyde is acrolein, and the unsaturated carboxylic acid is acrylic acid.
